(19) United States
(12) Patent Application Publication
(10) Pub. No.: US 2014/0127768 A1
(43) Pub. Date: May 8, 2014

(54) SOLAR-ASSISTED VOLATILE FERMENTATION PRODUCTS PRODUCTION PROCESSES

(71) Applicant: SCALE BIOFUEL, APS, Aalborg (DK)

(72) Inventor: Dan Nilsson, Aalborg (DK)

(73) Assignee: SCALE BIOFUEL, APS, Aalborg (DK)

(21) Appl. No.: 14/117,790

(22) PCT Filed: May 18, 2012
(86) PCT No.: PCT/EP2012/002138

§ 371 Date: Jan. 16, 2014

Related U.S. Application Data
(60) Provisional application No. 61/512,217, filed on Jul. 27, 2011.
(60) Provisional application No. 61/487,492, filed on May 18, 2011.

(51) Int. Cl. C12P 7/16; C12P 7/06

(52) U.S. Cl. CPC C12P 7/16; C12P 7/06
USPC 435/158; 435/157; 435/160; 435/161; 435/294.1

(57) ABSTRACT

The present invention provides a method and apparatus for recapturing heat from a solar-assisted volatile fermentation product production process comprising harvesting a volatile fermentation product from a solar-assisted fermentation product production apparatus and utilizing a heat recovery apparatus for recapturing the heat produced during the solar-assisted fermentation product production process. The volatile fermentation product can be produced in an autotrophic organism or by a fermenting organism fermenting fermentable sugars from one or more sugar crops, starch-containing and lignocellulose-containing materials.

### TECHNICAL FIELD

The invention relates to solar-assisted processes for producing, processing and/or recovering volatile fermentation products and the byproducts therefrom, and methods for improving the process economics of the same.

### BACKGROUND OF THE INVENTION

The escalating cost of fossil fuels and the increased world demand for such fuels have generated a market shift to the development and use of alternative fuels for energy needs such as transportation, heating, and electricity generation.

The most common biofuel currently used for the transportation sector is fuel ethanol. The primary sources of fuel ethanol being produced today are corn (US) and sugarcane (Brazil), but the preferred source of the future is likely cellulosic biomass. Regardless of the source of fermentable sugars for generating fuel ethanol, the capital expenditure (CAPEX) required to building a commercial plant to produce 50 to 100 million gallons of fuel ethanol per year is estimated at $100M-$200M USD. Thus, a large financial hurdle exists, in even the most developed countries, to substantially increase fuel ethanol production due to the CAPEX required to build new production facilities. In lesser developed countries, this CAPEX requirement essentially eliminates the possibility of those countries producing domestic fuel ethanol.

Butanol is another alternative fuel that can be a replacement for traditional gasoline. Butanol has many advantages since it is a fuel that can be utilized in automotives without any major engine modifications and can be shipped through existing fuel pipelines. In addition, butanol has a high energy content (110,000 Btu per gallon for butanol vs. 84,000 Btu per gallon for ethanol) whereas gasoline is about 115,000 Btu per gallon. Butanol is six times less “evaporative” than ethanol and 13.5 times less evaporative than gasoline. However, there has been little to no effort to promote butanol as an alternate fuel because of historically low yields and low concentrations of butanol compared to those of ethanol. Butanol can be manufactured from petroleum. However, butanol was historically manufactured from corn and molasses in a fermentation process that also produced acetone and ethanol known as an ABE (acetone, butanol, ethanol) fermentation. As demand for butanol increased, production by fermentation declined mainly because the price of petroleum dropped below that of sugar. Further, comparing ABE fermentation yield to that of the yeast ethanol fermentation yields, the yeast process for producing ethanol yields 2.5 gallons of ethanol from a bushel of corn at concentrations of 10-20%, whereas ABE fermentation yields 1.3 gallons of butanol 0.7 gallons of acetone and 0.13 gallons of ethanol per bushel of corn with butanol concentrations of only 1-2%.

A vast number of processes for producing volatile fermentation products, especially biofuel products such as ethanol and butanol, by fermentation of sugars derived from starch-containing and/or lignocellulose-containing material are known in the art. Processes for producing biofuel products, as well as other volatile organic compounds by fermentation processes or otherwise, often involve a recovery step wherein the volatility of the compound to be recovered is utilized to assist in the recovery process. This recovery process involves a substantial energy input that can be the most costly component of the entire production process.

Distillers grains (DGs) are a byproduct of fuel ethanol production. They have a very long history of being fed to livestock and are a source of additional revenue to the ethanol producers and/or the farmers that source the feedstocks for ethanol production. However, processing these distillers grains following the fermentation process is very expensive. Typically the energy required for dewatering and drying the DGs is one of the largest energy inputs into a traditional ethanol production process. Decreasing the amount of energy necessary for these drying and dewatering processes is highly desired.

Solar-assisted methods for the production and recovery of volatile compounds have been described, for example, in WO/2011/092638. These methods employ solar energy to provide energy in the form of heat to the production processes. However, much if not all of this heat is later rejected during the production process as “waste” heat.

Recovering and reusing rejected heat from commercial processes is generally known as waste heat recovery. The potential of waste heat recovery is dependent upon many factors including the quality of the waste heat stream, the applications available for using the recaptured heat, and the types of heat recovery equipment available. Conventionally, high grade waste heat generally above 500° C. is most suitable for waste heat recovery, but recovery from low-grade waste heat below 200° C. can provide net economic advantages. Thus, methods that employ solar energy input and waste heat recapture are desirable for improving overall process economics in volatile organic compound production processes.

### SUMMARY OF THE INVENTION

In one aspect, the present invention provides a method for recapturing heat from a solar-assisted volatile fermentation product production process comprising harvesting a volatile fermentation product from a solar-assisted feimentation product production apparatus, and utilizing a heat recovery apparatus for recapturing the heat produced during the solar-assisted fermentation product production process.

In another aspect, the present invention provides an apparatus for recapturing heat from a solar-assisted fermentation product production process comprising a heat recovery apparatus and a solar-assisted fermentation product production apparatus.

### BRIEF DESCRIPTION OF THE DRAWINGS

FIG. 1 is a schematic of one embodiment of an apparatus of the present invention.

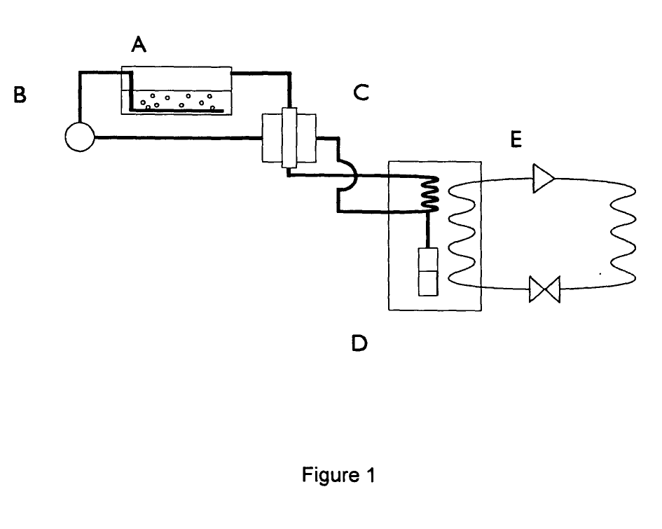

FIG. 2 represents temperature changes of various mediums during a two day simulation experiment for harvesting ethanol in one embodiment of an apparatus of the present invention.

FIG. 3 represents temperature changes of various mediums during a three day simulation experiment for harvesting ethanol in one embodiment of an apparatus of the present invention

FIG. 4 represents temperature changes of various mediums during a two day simulation experiment for harvesting sec-butanol in one embodiment of an apparatus of the present invention.

FIG. 5 represents temperature changes of various mediums during a two day simulation experiment for harvesting n-butanol in one embodiment of an apparatus of the present invention.

FIG. 6 represents temperature changes of various mediums during a two day simulation experiment for harvesting ethanol in the presence of linseed oil in one embodiment of an apparatus of the present invention.

FIG. 7 represents a schematic of an embodiment of the present invention wherein one or more secondary vessels are employed for processing byproducts of the method of the present invention.

### DETAILED DESCRIPTION OF THE INVENTION

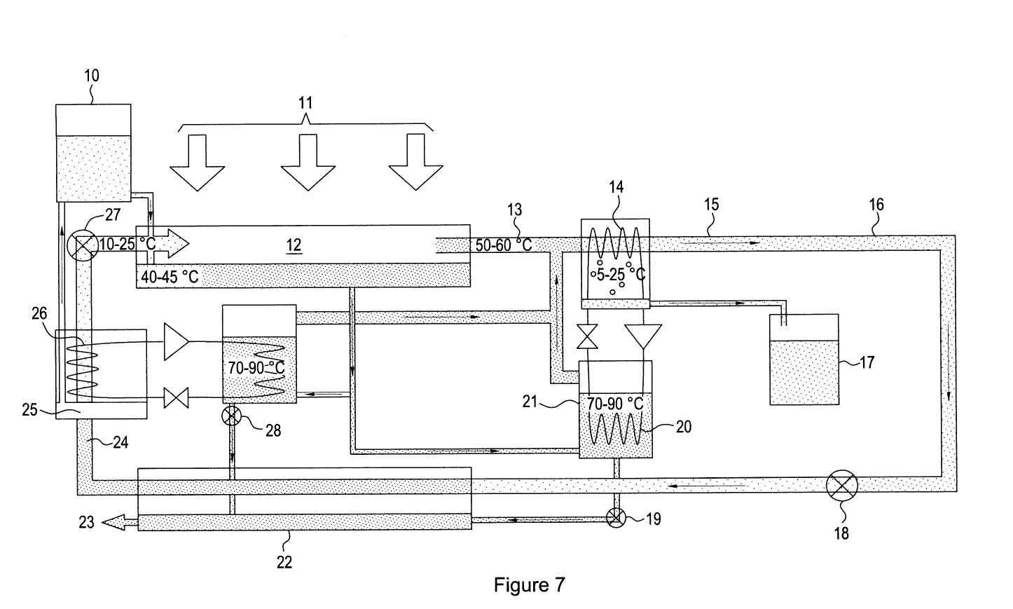

The following table provides the key for the reference numerals provided in FIG. 7.

FIG. 7

| | |
|---|---|
| 10 | Mixing vessel Raw material |
| 11 | Solar insolation 550 kWh day⁻¹ |
| 12 | Fermentation vessel |
| 13 | 425 kWh day⁻¹ hot moist air |
| 14 | Cooling loop |
| 15 | Cool “dry” air out |
| 16 | Cold air to thin stillage vessel |
| 17 | Condensate |
| 18 | Air pump |
| 19 | Liquid pump |
| 20 | Heating Loop |
| 21 | Fermentate boiler, evaporator; distillation |
| 22 | Thin stillage vessel |
| 23 | Wet distillers grain |
| 24 | 425 kWh day⁻¹ hot moist air |
| 25 | Water |
| 26 | Cooling loop |
| 27 | Air Pump |
| 28 | Liquid Pump |

Disclosed herein is a method for recapturing heat from a solar-assisted volatile fermentation product production process comprising harvesting a volatile fermentation product from a solar-assisted fermentation product production apparatus, and utilizing a heat recovery apparatus for recapturing the heat produced during the solar-assisted fermentation product production process. In one embodiment, the volatile fermentation product is produced in an autotrophic organism or by a fermenting organism fermenting fermentable sugars from one or more sugar crops, starch-containing and lignocellulose-containing materials.

The method of the present invention takes advantage of natural solar energy to produce and/or harvest volatile fermentation products utilizing an apparatus wherein the fermentation product can be partially or completely produced in a main vessel, the fermentation product is evaporated as a gas into the enclosed headspace of the main vessel to produce a vapour stream, and the vapour stream comprising the gas-phase fermentation product is condensed into a liquid-phase fermentation product condensate in a separate condensing unit. In one embodiment, the fermentation product is produced outside of the main vessel, and is placed into the main vessel for evaporation and/or condensing.

In one embodiment, the present invention provides a method for recapturing heat from a solar-assisted fermentation product production process comprising placing into an enclosed main vessel a fermentation medium comprising fermentable sugars and a fermenting organism capable of fermenting such fermentable sugars into a volatile fermentation product, fermenting the fermentable sugars with the fermenting organism to produce the fermentation product, evaporating the fermentation product as a gas into a headspace above the fermentation medium within the main vessel to form a vapour stream, and condensing the vapour stream comprising the gas-phase fermentation product into a liquid-phase fermentation product condensate in a condensing unit, wherein the condensing unit comprises one or more heat recovery apparatus. In a preferred embodiment, a heat source for the heat recovery apparatus is the vapour stream comprising the gas-phase fermentation product. In another preferred embodiment, a primary or secondary heat source for the heat recovery apparatus is a conventional heat source such as electricity, wood or other biomass-based, or liquid fuel-based heat source.

In one embodiment, the fermentable sugars and the fermenting organisms are combined prior to placing them into the main vessel. For example, such sugars and organisms can be combined in a mixing tank located proximately or remotely to the main vessel. The mixing tank can be further used as a propagation tank for propagating the fermenting organisms. The mixing tank can be subject to temperature and/or pressure regulation in accordance with the requirements for propagation of the selected fermenting organisms and can further be used as a fermentation vessel for producing the fermentation product. The liquid material in the mixing tank comprising the fermentable sugars and fermenting organisms can form the fermentation medium that is placed into the main vessel for further fermentation and/or evaporation.

The method, according to the present invention, is carried out in an apparatus comprising an enclosed main vessel located outdoors in a manner wherein the main vessel is exposed to direct sunlight, and a condensing unit. The main vessel comprises an upper portion and a lower portion. The lower portion of the main vessel contains the fermentation medium and the upper portion of the main vessel contains the headspace above the fermentation medium. The upper portion of the main vessel can be made of clear plastic or any other transparent, translucent or solid material that allows sunlight or solar energy to enter the main vessel. In one embodiment, the upper portion of the main vessel may further comprise one or more devices, covers, or other shade producing or insulating members to reduce the amount of solar energy entering the main vessel and/or to reduce heat loss from inside the main vessel. The lower portion of the main vessel containing the fermentation medium can be made of any material suitable for containing the liquid medium. The lower portion of the main vessel can be located on the surface of the ground, elevated above the surface of the ground, or located partially or full below the surface of the ground. The height above or depth below the ground surface of the lower portion of the main vessel depends upon the amount of insulation or cooling desired for the fermentation medium contained in the lower portion of the main vessel. The main vessel contains one or more inlets and discharges for introducing and removing solids, liquids and/or gases. The condensing unit of the apparatus is a separate unit from the enclosed main vessel and comprises one or more heat recovery apparatus capable of recapturing heat from the solar-assisted fermentation product production process. The condensing unit can be any structure or apparatus suitable for condensing the gas-phase fermentation product into a liquid-phase fermentation product condensate and capable of collecting the liquid-phase fermentation product condensate.

The main vessel and the condensing unit are connected by one or more means for transporting gases, liquids, solids, or a combination thereof, to and from the main vessel and the condensing unit. Such means include but are not limited to pipes or tubes made of one or more materials suitable for transporting such gases, liquids, solids or combinations thereof. In one embodiment, the lower portion of the main vessel comprises at least one discharge for removing all or part of the fermentation medium from the main vessel. The fermentation medium removed from the main vessel may contain solids and can be transported by any suitable means such as a tube or pipe, optionally with the aid of a pump, to a centrifuge where some or all of the solids can be removed from the fermentation medium. Some or all of the removed solids can be further processed for incorporation into farm feed such as wet distiller's grains or distiller's dried grains (DDGs) for cattle or other livestock feed. All or part of the liquid portion of the fermentation medium, and some or all of the solids, can be transported directly back to the main vessel, or to a mixing tank. Alternatively or in addition, part or all of the fermentation medium, with or without solids, can be transported from the main vessel to a boiler or evaporator for further fermentation product evaporation. The boiler or evaporator can be further connected to the condensing unit so as to condense the gas-phase fermentation product produced in the boiler or evaporator into liquid phase condensate in the condensing unit. In one embodiment, the boiler or evaporator is a component of the condensing unit. In another embodiment, the boiler or evaporator is further connected to one or more secondary vessels comprising the same or similar characteristics as the main vessel. The secondary vessel comprises at least two inlets and at least two discharges for receiving and discharging gases, liquids, solids, and combinations thereof. The secondary vessel can receive all or part of the fermentation medium, with or without solids, from which the fermentation product has been partially or substantially removed. The fermentation medium, with or without solids can be held in the secondary vessel, wherein the secondary vessel is exposed to direct sunlight. In the secondary vessel the fermentation medium with or without solids can be heated such that the water evaporates into the headspace of the secondary vessel as gas-phase water vapor, and the gas-phase water vapor can further be removed from the secondary vessel. One embodiment of an apparatus of the present invention employing one or more secondary vessels is depicted in the schematic of FIG. 7.

In one embodiment, a secondary vessel is employed to purify any portion or substantially all of the water captured from the method of the present invention. Along with capturing the fermentation product in a method of the present invention, water is also captured. The water can be purified for human consumption or for reusing in a method of the present invention or in some other industrial process.

In one embodiment, the upper portion of the main vessel comprises at least one inlet for receiving gases such as air, CO₂, or some combination thereof, and at least one discharge for removing the vapour stream comprising the gas-phase fermentation product from the upper portion of the main vessel for condensing into a liquid-phase fermentation product condensate in the condensing unit. The condensing unit comprises at least one inlet for receiving gas-phase fermentation product from the main vessel and/or the boiler or evaporator, and one or more means for collecting liquid such as the liquid-phase fermentation product condensate. In one embodiment, the condensing unit further comprises at least one discharge in which gases such as air, CO₂ or a combination thereof is directly or indirectly returned to the main vessel. The liquid-phase fermentation product condensate can optionally be removed from the condensing unit through a discharge in the condensing unit for further fermentation product enrichment, such as through further distillation by any means, or storage. Such further distillation or storage can occur proximately and/or remotely to the main vessel and/or the condensing unit.

The condensing unit further comprises one or more apparatus for recovering the waste heat produced during the solar-assisted fermentation product production process. Various forms of heat recovery processes and apparatus are known in the art. Selection of the appropriate process and/or apparatus for recovery of waste heat in the method of the present invention is, among other factors, dependent upon the temperature of the waste heat to be recaptured. Waste heat quality is typically directly related to the temperature of the heat being rejected from a system. Generally, the quality of the waste heat increases as the temperature of the waste heat increases. However, low grade waste heat (typically less than 200° C.) can provide both direct and indirect economic benefits.

Typical uses of low grade waste heat include supplemental or preheating of air or liquids for a commercial process or for general heating of hot water for industrial or household use. However, low grade waste heat can also be used for generating power utilizing a Rankine cycle, an organic Rankine cycle, or modifications thereof. For example, U.S. Pat. No. 7,278,264 discloses various processes for converting low grade heat sources into power, and such disclosure and processes are herein incorporated by reference.

Any apparatus capable of recapturing the waste heat generated by the solar-assisted fermentation product production process is suitable for the method of the present invention and includes conventional heat pumps, and other heat and moisture transfer apparatus such as those described in U.S. Pat. No. 4,952,283, U.S. Pat. No. 5,689,962, and Wongsuwan et al., Applied Thermal Engineering 21 (2001) 1489-1519; each herein incorporated by reference. In one embodiment, a heat recapture system suitable for the present invention comprises a machine or device that is capable of transferring lower temperature heat from a heat source to a higher temperature heat sink by using mechanical work. In such an embodiment, the machine or device can comprise a working liquid or gas, a condenser, an expansion valve, an evaporator, and a compressor wherein the compressor is a pump that can pressurize the working liquid or gas. The pressurized working liquid or gas can flow from the compressor to the condenser where the heat from the heat source is released into the heat sink. The working liquid or gas can then pass through the expansion device to the evaporator where heat from the heat source can be collected by the working liquid or gas which then can flow back to the compressor. In another embodiment, the heat recapture system comprises a heat exchanger, such as and air to air or air to liquid heat exchanger, and one or more enclosures capable of capturing the heat released by the heat exchanger. In a further embodiment of the present invention, the heat source is the vapour stream comprising the gas-phase fermentation product from the upper portion of the main vessel of the solar-assisted fermentation product production process. In another embodiment, the heat sink is any recipient of the heat released from the condenser such as water, air or any other substance in need of heating. In a further embodiment, the apparatus capable of recapturing heat has an auxiliary heat source that can be employed alone or in combination with the heat source from the vapour stream. In a further embodiment, the entire apparatus can be adapted for alternative uses such as water purification.

In a solar-assisted process for producing volatile fermentation products in an apparatus according to the present invention, from time to time it may be necessary to control the amount of fermentation medium in the main vessel by removing some of the fermentation medium from the main vessel. The fermentation medium that can be removed from the main vessel contains the fermentation product and may contain solids capable of being converted to value-added products. In one embodiment, the recaptured waste heat is used, alone or in combination with traditional heat sources, to heat all or part of the removed fermentation medium in a boiler or evaporator to recover gas-phase fermentation product in the form of a vapour stream from the removed fermentation medium. The vapour stream comprising the gas-phase fermentation product can be returned to the condensing unit for condensing the gas-phase fermentation product into liquid-phase fermentation product condensate, and the waste heat from the boiler or evaporator can further be recaptured by one or more methods disclosed herein. The solids contained in the removed fermentation medium can be recovered to produce wet distiller's grains or distiller's dried grains. Thus, in one embodiment, the recaptured heat is used to dry the solids recovered from the removed fermentation medium.

In particular embodiments of the present invention, the heat recaptured by the heat recovery system can also be used for one or more of the following purposes: heating the fermentation medium in the main vessel; heating the cooled “dry” air released from the condensing unit prior to returning the air to the main vessel; heating a fermentable sugars solution to evaporate off excess water; and heating products in need of drying such as lumber, wood chips, water and food products.

In one embodiment the method of the present invention employs the natural solar energy to elevate the temperature in the headspace of the upper portion of the main vessel. During the daylight hours, the headspace heats up relatively quickly. Not being bound by any particular theory, such temperature increase in the headspace causes an increase in the rate of evaporation of the fermentation product in the fermentation medium in the lower portion of the main vessel to produce a vapour stream comprising the gas-phase fermentation product in the headspace of the main vessel. In one embodiment, the vapour stream comprising the gas-phase fermentation product is forced out of the headspace by a pump into the condensing unit so the gas-phase fermentation product entering the condensing unit is rapidly cooled to form liquid-phase fermentation product condensate, and the heat from the vapour stream is recaptured by one or more apparatus capable of waste heat recapture.

The maximum temperature achieved in the headspace of the main vessel, and hence the maximum temperature of the waste heat vapour stream, depends, among other things, on the amount of solar energy that enters the vessel during daylight hours and the amount of heat retained in the main vessel during the non-daylight hours. In one embodiment of the present invention, the method comprises heating the headspace of the main vessel during daylight hours to a maximum temperature between about 4° C. and about 85° C. In another embodiment, the headspace of the main vessel is heated to a maximum temperature between about 25° C. and about 70° C. during daylight hours. In a further embodiment, the headspace of the main vessel is heated to a maximum temperature between about 40° C. and about 65° C. during daylight hours.

Due to the rising and setting of the sun, the amount of solar energy entering the main vessel will fluctuate during a 24 hour period. Therefore, in one embodiment, the temperature of the headspace and the temperature of the fermentation medium will fluctuate over a 24 hour period. In an effort to maintain or decrease the natural fluctuation of the temperature of the headspace or the temperature of the fermentation medium during the method of the present invention, one or more temperature regulation methods can be employed. In one embodiment, the depth of the fermentation medium can be adjusted in order to decrease the amount of temperature fluctuation in the fermentation medium during a 24 hour period or any shorter or longer period of time. In another embodiment, the rate that the gases are pumped through the apparatus can be increased or decreased to decrease the temperature fluctuation in the headspace during a 24 hour period or any shorter or longer period of time.

Microorganisms such as yeast and some bacteria are capable of fermenting sugars to produce fermentation products. Sugars that bacteria and yeast are capable of directly or indirectly converting into fermentation products are herein referred to as “fermentable sugars.” For purposes of the present invention, examples of fermentable sugars include, but are not limited to, sucrose, glucose, fructose, xylose, mannose, and galactose, or any saccharide typically containing five or six carbon atoms that can be directly or indirectly fermented into fermentation products by certain fermenting organisms.

The concentration of fermentable sugars in the fermentation medium can vary according to the fermenting organism employed as well as the desired fermentation product. In one embodiment of the method of the present invention, the fermentable sugars are at a concentration of about 2-50% w/v in the fermentation medium. In one embodiment, concentrations of about 10% to about 50% are suitable for fermentation organisms that produce ethanol. In another embodiment, concentrations of about 2% to about 15% are suitable for fermenting organisms that produce butanol.

### Fermentation Products

Many products can be produced in fermentation processes. However, for purposes of the present invention, the fermentation products produced and harvested by methods of the present invention are volatile fermentation products. Generally, volatile compounds are compounds with boiling points below 150° C. and vapor pressures of greater than 0.1 mm Hg. Examples of such products include, but are not limited to, ethanol, 1-propanol, 2-propanol, n-butanol, sec-butanol, iso-butanol, acetone, acetic acid, butyric acid, acetaldehyde, acetoin, 2,3-butanediol, butanone, and flavouring compounds such as esters.

In accordance with the present invention, the volatile fermentation products may or may not be produced by a fermentation process. For example, autotrophic organisms are capable of producing the volatile fermentation product butanol. Butanol produced by such organisms is a volatile fermentation product in accordance with the present invention.

Fermentable Sugars from Sugar Crops

Certain sugar crops such as sugarcane, sugar beets, and sweet sorghum contain a large amount of fermentable sugars that can be fermented directly or indirectly into fermentation products by certain fermenting organisms. Fermentation products of particular interest are ethanol, n-butanol, sec-butanol, and iso-butanol. Sugar crops can readily be fermented by certain fermenting organisms to produce the desired fermentation products. For example, at the time of harvest, sugarcane generally contains about 90% sucrose and about 10% combined glucose and fructose. Such sugars are extracted from the sugarcane in the form of sugarcane juice. The juice, syrup, or the molasses produced as a byproduct of the process for producing sugar from sugarcane, can directly or indirectly be fermented into ethanol or butanol by certain fermenting organisms such as yeast and bacteria, respectively.

Fermentable Sugars from Starch-Containing Material

Production of fermentation products, such as ethanol and butanol, from starch-containing material is well-known in the art. Starch-containing materials for purposes of the present invention include, but are not limited to, corn, wheat, grain sorghum, barley, cassava, and potatoes. Generally two different kinds of processes are used to generate fermentable sugars from starch-containing material. The most commonly used process, often referred to as the “conventional process,” includes liquefaction of gelatinized starch at high temperature using typically a bacterial alpha-amylase, followed by saccharification carried out in the presence of a glucoamylase. Another well-known process, often referred to as a “raw starch hydrolysis” process (RSH) includes saccharifying granular starch below the initial gelatinization temperature typically in the presence of an acid fungal alpha-amylase and a glucoamylase. In both the conventional and raw starch processes, saccharification can be carried out separately or simultaneously with fermentation. According to the present invention, saccharification of gelatinized starch can occur prior to fermentation. Preferably, according to the present invention, if starch-containing material is the source of fermentable sugars for the present invention, the fermentable sugars can be generated utilizing a raw starch hydrolysis process prior to or concurrent with fermentation. The RSH can occur in the same main vessel as the fermentation and/or evaporation, or the RSH can occur in a separate vessel located proximately or remotely to the main vessel. Hydrolysis of starch-containing materials by either method described above is well known in the art and is described, for example, in WO/2010/022045.

Fermentable Sugars from Lignocellulose-Containing Biomass

In order to obtain fermentable sugars from lignocellulose-containing biomass, the cellulose and hemicellulose components of the biomass must be broken down into fermentable sugars by hydrolysis. Examples of lignocellulose-containing biomass for purposes of the present invention, include but are not limited to corn fiber, rice straw, pine wood, wood chips, bagasse, paper and pulp processing waste, palm oil waste, corn stover, corn cobs, hard wood such as poplar and birch, soft wood, cereal straw such as wheat straw, rice straw, switch grass, Miscanthus, rice hulls, municipal solid waste (MSW), industrial organic waste, office paper, or mixtures thereof.

Methods for producing fermentable sugars from lignocellulosic biomass are well known in the art and such methods typically combine one or more processes such as pretreatment and/or acid or enzymatic hydrolysis. Methods for obtaining fermentable sugars from lignocellulose-containing materials are described, for example, in WO/2010/039812. Other suitable sources include lignocellulose-derived sugars by radical chain reaction chemistry such as GAF catalysis of lignocellulosic material by Georgia Alternatives Fuels, LLC, Georgia, U.S.A.

In one embodiment, the fermentable sugars are obtained from one or more sugar crops such as sugarcane, sugar beets, and sweet sorghum. In another embodiment, the fermentable sugars are obtained from starch-containing materials such as corn. In another embodiment, the fermentable sugars are obtained from one or more lignocellulose-containing materials such as switch grass and bagasse. In a further embodiment, the source of fermentable sugars is a concentrated sugar feedstock such as feedstock from Sweetwater Energy, Inc., Rochester, N.Y., U.S.A. or Virdia Redwood City, Calif., U.S.A.

### Fermenting Organisms

The term “fermenting organism” refers to any organism, including bacterial and fungal organisms, such as yeast and filamentous fungi, suitable for producing fermentation products.

### Ethanol as a Fermentation Product

Wherein ethanol is the desired fermentation product, suitable fermenting organisms according to the invention are able to ferment, i.e., convert sugars, such as sucrose, glucose, fructose, maltose, xylose, mannose and/or arabinose, directly or indirectly into ethanol. Examples of fermenting organisms include fungal organisms, such as yeast. Contemplated strains of yeast include strains of the genus Saccharomyces, in particular a strain of Saccharomyces cerevisiae or Saccharomyces uvarum; a strain) of Pichia, in particular Pichia stipitis or Pichia pastoris; a strain of the genus Candida, in particular a strain of Candida utilis, Candida arabinofermentans, Candida diddensii, Candida sonorensis, Candida shehatae, Candida tropicalis, Candida digboiensis, Candida thermophila, or Candida boidinii. Other contemplated yeast includes strains of Hansenula, in particular Hansenula polymorpha or Hansenula anomala; strains of Kluyveromyces, in particular Kluyveromyces marxianus or Kluyveromyces fagilis, and strains of Schizosaccharomyces, in particular Schizosaccharomyces pombe.

Contemplated bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1 L1 (Appl. Microbiol. Biotech. 77: 61-86) and Thermoanarobacter ethanolicus, Thermoanaerobacter thermosaccharolyticum, or Thermoanaerobacter mathranii. Strains of Lactobacillus are also envisioned as are strains of Corynebacterium glutamicum R, Bacillus thermoglucosidaisus, and Geobacillus thermoglucosidasius.

In connection with especially fermentation of lignocellulose derived fermentable sugars, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp. that are capable of fermenting C5 sugars include the ones concerned in, e.g., Ho et al., 1998, Applied and Environmental Microbiology, p. 1852-1859 and Karhumaa et al., 2006, Microbial Cell Factories 5:18, and Kuyper et al., 2005, FEMS Yeast Research 5, p. 925-934.

Certain preferred fermenting organisms include Candida thermophila as described by Shin et al., Int J Syst Evol Microbiol, 51: 2167 (2001); a modified Bacillus strain as described in U.S. Pat. No. 7,691,620; one or more Geobacillus strains as described in Tang et al., Biotechnology and Bioengineering, 102: 1377-1386 (2009); Kluyveromyces marxianus as described in Babiker et al., Appl Microbiol Biotechnol (2010) 85:861-7; and the ethanol producing mesophilic and thermophilic organisms described in WO2006/117536, WO2008/038019, WO2008/141174, WO2009/022158, WO2010/052499, and those described by RE Cripps et al., Metabolic Engineering 11 (2009) 398-408.

### Butanol as a Fermentation Product

Wherein butanol is the desired fermentation product, especially suitable fermenting organisms include Clostridium such as Clostridium acetobutylicum, Lactobacillus strains such as Lactobacillus buchnerii, Lactobacillus lactis, Lactobacillus delbrueckii, and Lactobacillus brevis, Pseudomonas strains such as Pseudomonas putida, Bacillus strains such as Bacillus subtilis, Saccharomyces strains such as Saccharomyces cerevisiae, Zymomonas strains such as Zymomonas mobilis, Candida strains such as Candida acidothermophilum and Candida sonorensis, Pachysolen strains such as Pachysolen tannophilus, Pichia strains such as Pichia guilliermondii and Pichia methanolica, Escherichia strains such as Escherichia coli, and genetically modified strains of the same as described, for example, in V. Garcia et al., Renewable and Sustainable Energy Reviews 15 (2011) 964-980; Baez et al., Appl Microbiol Biotechnol (2011) 90:1681-1690; Sakuragi et al., and Journal of Biomedicine and Biotechnology (2011) Volume 201, Article ID 416931, 11 pages. In accordance with the present invention, butanol such as n-butanol can also be produced from CO₂ by genetically modified cyanobacterium, such as the Synechococcus elongatus described by E. I. Lan and J. C. Liao; Metabolic Engineering 13 (2011) 353-363.

For purposes of the present invention, the term “thermophile” means a microorganism that grows optimally at temperatures between about 40° C. and about 85° C., yet also includes organisms that can grow or withstand temperatures as low as about 4° C. and as high as about 105° C. According to the method of the invention, selection of the fermenting organism depends primarily on the source of fermentable sugars, the temperature range at which fermentation is carried out, and the level of ethanol tolerance of the fermenting organism. One skilled in the art can readily select a fermenting organism for use in the present invention based on these and other parameters understood by those skilled in the art. According to the method of the present invention, the fermenting organism can be a naturally occurring organism or a genetically modified organism.

In one embodiment one or more fermenting organisms are added to the fermentation medium so that the viable fermenting organism, such as yeast, count per ml of fermentation medium is in the range from 10⁵ to 10¹², preferably from 10⁷ to 10¹⁰, especially about 5×10⁷. Commercially available yeast for producing ethanol include, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

Fermenting organisms used in the methods of the present invention can be affected negatively by the accumulation of the desired fermentation product in the fermentation medium. Therefore, it is often desirable to control the amount of fermentation products in the fermentation medium in order to maintain a continuous fermentation process or at least preserve the fermenting organisms for continued use in a fermentation process. The amount of fermentation product in the fermentation medium can be regulated by one or more means of concentration or dilution. For example, as the fermentation product evaporates out of the fermentation medium into the headspace of the main vessel, the concentration of the fermentation product in the fermentation medium typically decreases. The concentration of the fermentation product in the fermentation medium may also be decreased, perhaps only temporarily, with the addition of more fermentable sugars to the fermentation medium. After the fermentable sugars are added to the fermentation medium, the fermenting organisms can ferment the sugars into more fermentation product, thus increasing the concentration of the fermentation product in the fermentation medium. Thus, in one embodiment, additional fermentable sugars can be added to the fermentation medium in the main vessel, in a continuous or batch fashion, at a rate suitable for the fermenting organisms to continue to produce additional fermentation product as the fermentation product is being removed from the fermentation medium by evaporation into the headspace of the main vessel. In a preferred embodiment, fermentable sugars can be added and fermentation product can be removed, such that the entire fermentation process can be generally run continuously, Based on the tolerance of the fermenting organism for the fermentation product being produced, one skilled in the art can determine the desired range or ranges of fermentation product concentrations to be maintained in the fermentation medium during the fermentation process.

Methods for controlling the concentration of the desired fermentation product in the fermentation medium during a fermentation process are generally known as extractive fermentation methods. Extractive fermentation is an in situ solvent extraction process utilized during the fermentation process to extract the fermentation product from the fermentation medium without disrupting the further production of the desired fermentation product. Some of the most common processes utilize oleyl alcohol, decanol, and polypropylene glycol for extracting butanol. Many of these processes today have not been widely employed at a commercial scale since the cost to do so is generally high. More suitable methods for extractive fermentation on a commercial scale may include in situ product recovery as described in US2011/0312044, and methods that employ different types of oils that are compatible with the miscibility of the desired fermentation product to be extracted. See, for example, Ishizaki et al., J Biosci Bioeng, 87(3), 352-56 (1999) and “Separating Ethanol From Water Via Differential Miscibility,” Renaldo V. Jenkins of Langley Research Center, Journey to Forever, Online Biofuels Library, each incorporated herein by reference to the extent that they teach extractive fermentation and fermentation product separation. In one embodiment, one or more oils in which the desired fermentation product is miscible in, is added to the fermentation product production process. For example, in one embodiment, crude palm oil can be added during the fermentation product production process wherein the fermentation product comprises butanol. In another embodiment, castor or linseed oil can be added during the fermentation product production process wherein the fermentation product comprises ethanol. The oil can be added prior to, during, or after the fermentation medium is added to the main vessel, and it can be mixed into the fermentation medium or layered on top of the fermentation medium.

The addition of one or more oils to the fermentation medium may also assist in the recovery and/or increase the concentration of the fermentation product in the condensate produced by a method of the present invention. Not being bound by any particular theory, it is believed that the desired fermentation product is partially or fully extracted from the aqueous fermentation medium by the oil. Thus, it is believed that less water and more of the desired fermentation product will make up the gas-phase vapour stream exiting the main vessel than without the addition of the oil. Hence, the concentration of the desired fermentation product in the condensate may be higher than if the oil is not employed during the process. Therefore, the need for further distillation of the fermentation product may be reduced or eliminated completely.

As used herein, the phrase “fermentation media” or “fermentation medium” refers to the aqueous environment in which fermentation is carried out and comprises the fermentation substrate, that is, the carbohydrate source that is metabolized by the fermenting organisms to produce the fermentation product, and may include the fermenting organisms. The fermentation medium may further comprise nutrients and growth stimulators for the fermenting organisms. Nutrient and growth stimulators are widely used in the art of fermentation and include nitrogen sources, such as ammonia, vitamins and minerals, or combinations thereof. Following fermentation, the fermentation media or fermentation medium may further comprise the fermentation product such as ethanol or butanol. In methods of the present invention wherein the fermentation product is produced by a means other than fermentation, such as by an autotrophic organism, the phrase “fermentation media” or “fermentation medium” refers to the medium that the fermentation product is produced in and/or is harvested from.

While the foregoing description of the invention enables one of ordinary skill to make and use what is considered presently to be the best mode thereof, those of ordinary skill will understand and appreciate the existence of variations, combinations, and equivalents of the specific embodiments, methods, and examples herein. The invention is therefore not limited by the above described embodiments, methods, and examples, but by all embodiments and methods within the scope and spirit of the invention.

### EXAMPLES

### Materials and Methods

Fermentation mediums (simulated). For purposes of Examples 1-4, fermentation mediums were prepared as follows: In the experiments with ethanol solutions, 2.5 l of 96% v/v of denatured ethanol (Føtex, Aalborg, Denmark) was diluted to 20 l with tap water to give a final concentration of approximately 12% v/v ethanol. In the experiments with n-butanol and sec-butanol solutions, the fermentation medium consisted of 400 ml of n-butanol or sec-butanol (BDH Prolabo, GPR Rectapur, VWR Bie & Berntsen, Denmark) diluted to 20 l with tap water to give a final concentration of approximately 2% v/v. For Example 5, fermentation mediums were prepared as follows: 4 l of 93% v/v of denatured ethanol (Føtex, Aalborg, Denmark) was diluted to 20 l with tap water to give a final concentration of approximately 19% v/v ethanol. For all experiments, the fermentation medium was at a depth of approximately 20 cm in the fermentation vessel. The fermentation vessel dimensions were a height of 40 cm, a length of 40 cm and a width of 32 cm. The vessel was constructed of dark brown polyethylene on the sides and bottom, and the top of the vessel was closed with a 4 mm thick glass plate. The vessel was insulated on the sides and bottom with Rockwool 50 mm stone wool insulation (TUN No.: 16 24 527, Rockwool A/S, Hedehusene, Denmark) with an insulating capacity at 50° C. equal to 44 mW m⁻¹ K⁻¹. Before the vessel was closed with the top glass plate the headspace was filled with CO₂ from a 16 g CO₂ cartridge.

Air/CO₂ mixture was circulated through the fermentation medium of the vessel at a rate of approximately 10 to 15 m³/hour per m² of exposed surface area of the fermentation medium. In the present examples, the exposed surface area of the fermentation medium is 0.128 m², thus the corresponding air flow was approximately 21 to 32 Umin.

The vapour stream comprising the gas-phase fermentation product in the headspace above the fermentation medium was lead via a ⅜″ inner diameter hose into either an air to air plate heat exchanger connected in line to a hose condensing unit, or directly to the hose condensing unit. The air to air heat exchanger consisted of a plate heat exchanger (PHE) constructed from 16 aluminum plates contained in a unit being 34 cm×24 cm×4.5 cm supplied by Auto og Industri køler centret, Aalborg, Denmark, and two metal boxes attached to opposite sides of the PHE, each with the dimensions of 24 cm×23 cm×5 cm. The boxes were attached such that they formed a single enclosure around the plates of the PHE, thus contained the air flowing around the PHE. The PHE and the enclosure comprise the PHE+E. The enclosure was connected to two ⅜″ inner diameter hoses, one that lead the cooled air from the tube condenser to the enclosure and the other that lead the warmed air from within the enclosure to the fermentation vessel.

### Heat Recapture

Hose condensing unit: The vapour stream comprising the gas-phase fermentation product, either after passing through the PHE or coming directly from the fermentation vessel, was lead to a hose condensing unit. The hose condensing unit was constructed from a ⅜″ inner diameter plastic hose wrapped around a 10 L plastic bucket filled with cooled water. The distal end of the ⅜″ inner diameter hose of the hose condensing unit was connected to a Y shaped metal connector for gravity separation of the liquid condensate from the air flow. One outlet of the Y shaped plastic connector was connected with a ⅜″ inner diameter hose to a sealed 0.5 liter Bluecap bottle for collecting the liquid condensate. The remaining outlet of the Y connector was connected with a ⅜″ inner diameter plastic tube to the enclosure around the PHE. The plastic hose wrapped around the bucket, the Y connector, and the Bluecap bottle were contained within and cooled by a Matsui refrigerator (Matsui MUR1107WW, elgiganten, Aalborg, Denmark). The plastic hose wrapped around the bucket, the Y connector, the Bluecap bottle, and the refrigerator collectively comprise the hose condensing unit. The hose condensing unit captures heat from the vapour stream and via the heat pump of the refrigerator releases the heat to the room.

PHE+E: The enclosure around the PHE was further connected by a ⅜″ inner diameter plastic tube to a diaphragm air pump with a capacity of 33 L/min (B100, Charles Austen Pumps Ltd, Surrey, UK) regulated by a potentiometer regulated frequency converter (Motron FC750, Eltwin A/S, Risskov, Denmark). The pump causes the cool air leaving the hose condensing unit to enter the enclosure around the PHE and be warmed by passing over the PHE, and further be returned to the fermentation vessel after being warmed. Inside the fermentation vessel the air passes through the headspace of the main vessel (ethanol) or is bubbled through the fermentation medium (butanol) via three 25 cm long 32 mm PVC tubes containing 1 mm diameter holes spaced 1 cm apart along each tube. The three PVC tubes were connected end to end and placed in an s-shape at the bottom of the fermentation vessel.

### Solar Insulation

Six 60 W white light spot lamps (Concentra Spot R63, Osram Gmbh, Munich, Germany) hanging approximately 30 cm above the vessel provided radiant heat to the vessel. Based on the temperature increase in the fermentation medium and the vessel headspace and calculated evaporation heat of e.g. ethanol and water, it was determined that the average equivalent solar insolation of the white lights is roughly equivalent to 5.4 kwh/m²/day.

The ethanol concentration in the condensate was measured with an alcohol meter (Alkoholmeter tysk 30 cm, Vinøl Hobby, Frederiksberg, Denmark) calibrated to 0% v/v with tap water and 100% with denatured 93% v/v ethanol. Following each measurement the ethanol condensate was returned to the fermentation vessel.

The sec-butanol concentrations in the condensate and fermentation medium were measured by HPLC (K. Ohgren et al. Biomass and Bioenergy 30 (2006) 863-869).

### Example 1

### Day/Night Simulation with Ethanol without PHE+E

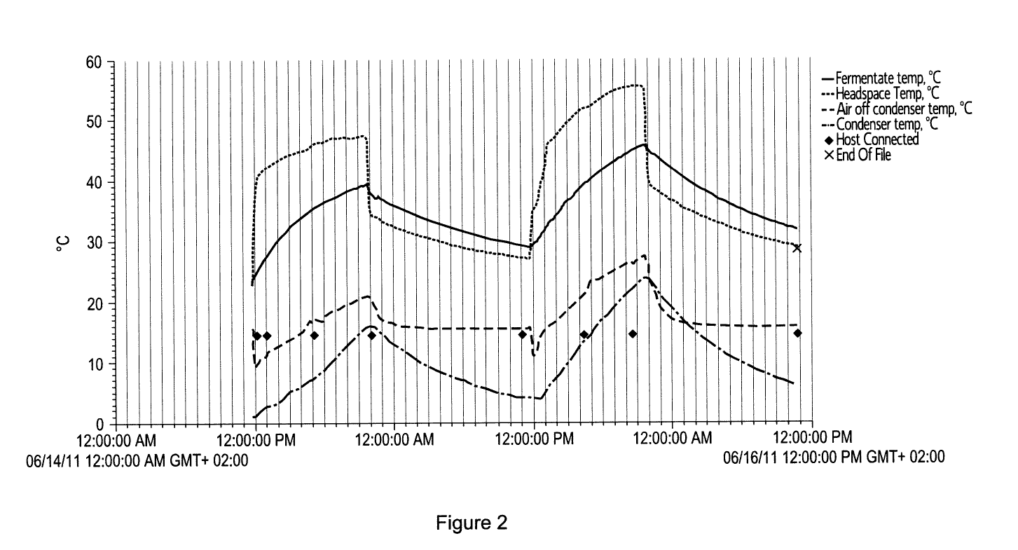

A 12% v/v ethanol solution was used to simulate an ethanol fermentation medium in the fermentation vessel as described above. The white light spot lamps were turned on for 10 hours and then turned off for 14 hours to simulate a natural day/night cycle. CO₂ was circulated through the headspace and condensing unit at a rate of approximately 15 m³/hour per m² exposed surface area of fermentation medium during the 10 hours of light. The exposed surface area of the fermentation medium was 0.128 m². The temperature of the fermentation medium, headspace, cooling water of the hose condensing unit and air going out of the hose condensing unit were measured periodically over two days and are displayed graphically in FIG. 2. Following each measurement of the ethanol condensate, the ethanol condensate was returned to the fermentation vessel. Therefore, the fermentation medium contained a relatively constant ethonol concentration of 12% v/v from day to day. The ethanol productivity was about 2.3 liter ethanol/day/m² at a concentration of about 30% v/v ethanol when measured on day two.

### Example 2

### Day/Night Simulation with Ethanol and with PHE+E

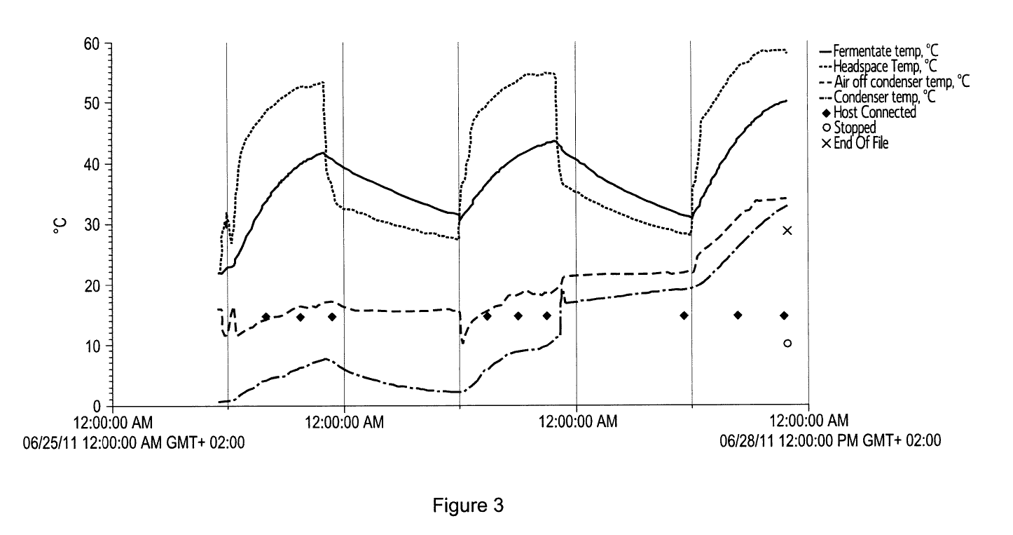

A 12% v/v ethanol solution was used to simulate an ethanol fermentation medium in the fermentation vessel as described above. The day/night simulation for Example 2 was as described in Example 1, except that during day one and two the PHE+E was incorporated into the setup to pass the vapour-phase ethanol through the PHE before entering the hose condensing unit, and further warming the cooled air that exited the hose condensing unit prior to returning it to the fermentation vessel by passing it through the enclosure around the PHE. Day three was used as a control wherein the lights were turned on for 10 hours, the PHE+E was removed and the external cooling for the hose condensing unit was turned off (i.e. the hose condensing unit was at room temperature). The temperatures that were measured are shown in FIG. 3.

The ethanol productivity at day two was about 3.4 l ethanol/day/m² at a concentration of about 35% v/v ethanol. The ethanol productivity was about 1.3 l ethanol/day/m² at a concentration of about 22% v/v ethanol on day three.

### Example 3

### Day/Night Simulation with 2-Butanol and with PHE+E

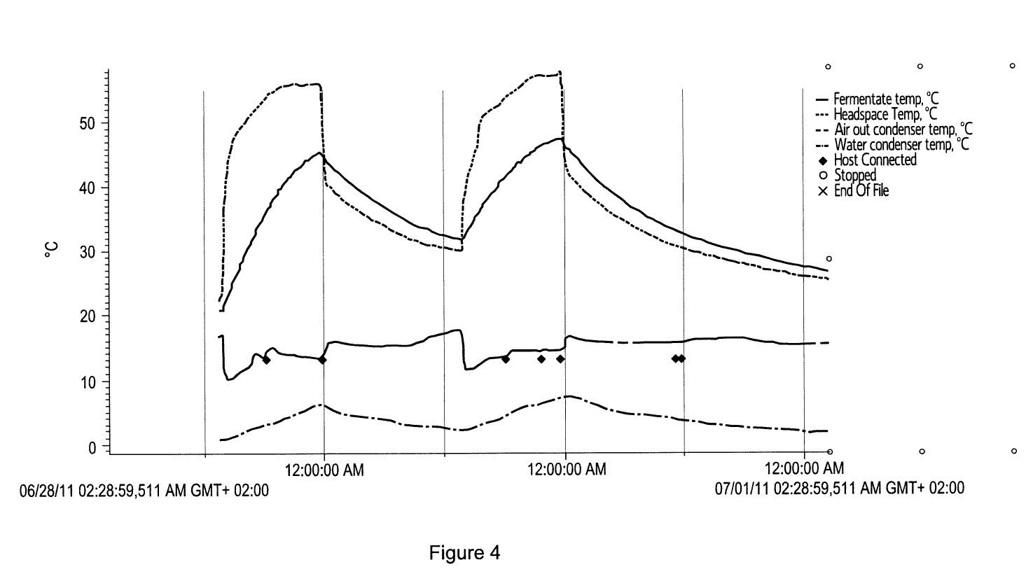

As in Example 2, the PHE+E and cooled hose condensing unit was used except a 2% v/v sec-butanol solution was used as the fermentation medium instead of the ethanol fermentation medium and the day/night simulation was concluded at the end of day two. The temperature readings during day three were taken during a time period wherein the lights were not turned on, the air pump was not running, and the external cooling of the hose condensing unit was turned off. The temperatures that were measured are shown in FIG. 4.

The butanol productivity at day two was about 0.77 liter 2-butanol/day/m² at a concentration of about 11.2% v/v sec-butanol.

### Example 4

### 2 Day/Night Simulation with n-Butanol and with PHE+E

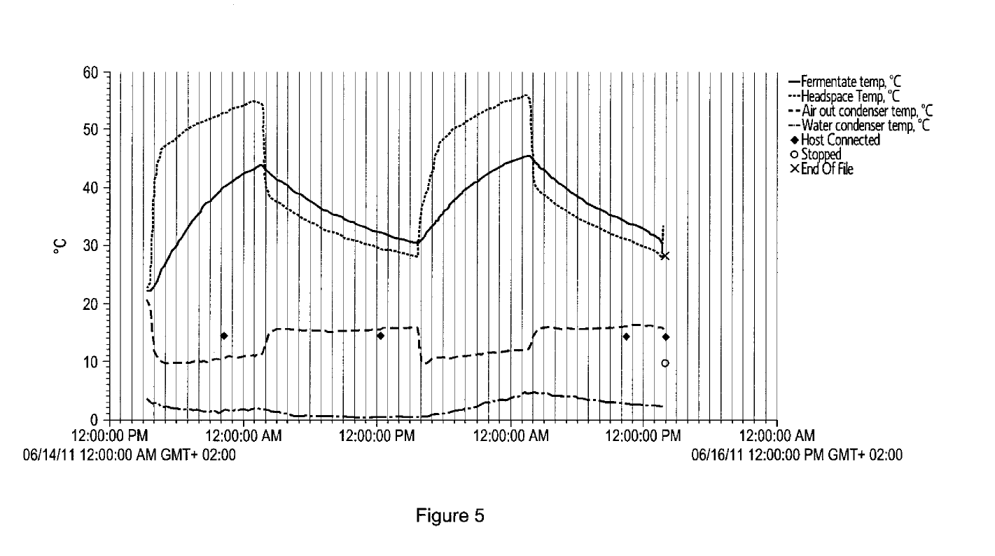

As in Example 3, the PHE+E and cooled hose condensing unit was used except a 2% v/v n-butanol solution was used as the fermentation medium instead of the sec-butanol fermentation medium and the day/night simulation was concluded at the end of day 2. The temperatures that were measured are shown in FIG. 5.

The n-butanol productivity of day 1 was about 0.61 liter condensate separated in 2 liquid phases consisting of an upper phase of about 75 ml 1-butanol and a lower phase about 0.54 liter 1-butanol saturated water giving a combined productivity of about 0.99 liter/day/m².

The n-butanol productivity of day 2 was about 0.8 liter condensate separated in 2 liquid phases consisting of an upper phase of about 17 ml 1-butanol and a lower phase about 0.79 liter 1-butanol saturated water giving a combined productivity of about 0.61 liter/day/m².

### Example 5

### Day/Night Simulation with Ethanol and Oil, without PHE+E

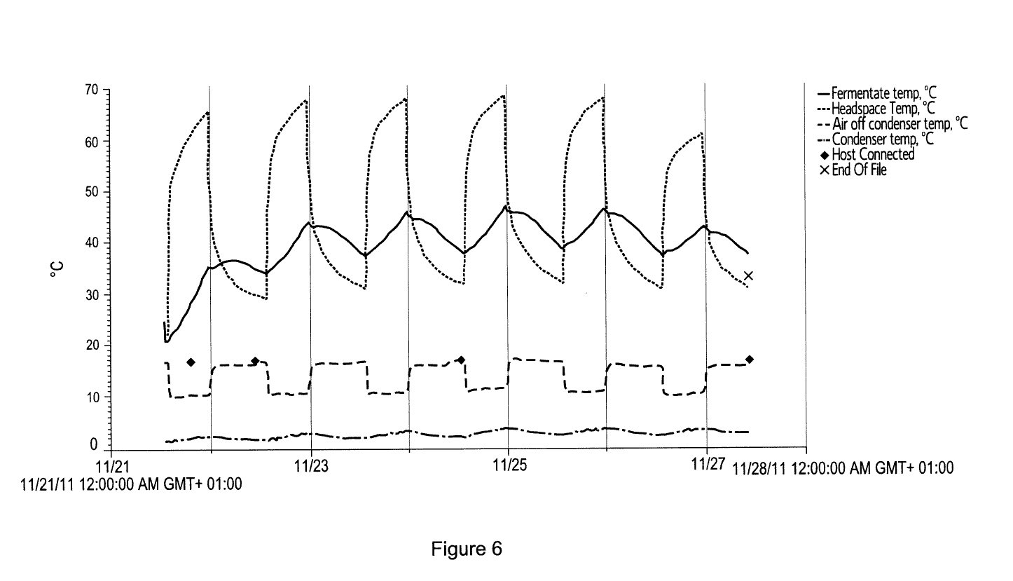

A 19% v/v ethanol solution was used to simulate an ethanol fermentation medium in the fermentation vessel as described above. To the fermentation medium was added 750 ml linseed oil to give an approximately 6 mm oil layer covering the surface of the fermentation medium. The white light spot lamps were turned on for 10 hours and then turned off for 14 hours to simulate a natural day/night cycle. CO₂ was circulated through the headspace and condensing unit at a rate of approximately 10 m³/hour per m² exposed surface area of fermentation medium during the 10 hours of light. The exposed surface area of the fermentation medium was 0.128 m². The temperature of the fermentation medium, headspace, cooling water of the hose condensing unit and air going out of the hose condensing unit were measured periodically over two days and are displayed graphically in FIG. 6. The highest ethanol productivity was obtained from day 3-6 about 0.5 liter ethanol/day/m² at a concentration of the condensate of about 69% v/v ethanol.

1. A method for recapturing heat from a solar-assisted volatile fermentation product production process comprising:
a. harvesting a volatile fermentation product from a solar-assisted fermentation product production apparatus; and
b. utilizing a heat recovery apparatus for recapturing the heat produced during the solar-assisted fermentation product production process.

2. The method according to claim 1, wherein the volatile fermentation product is produced in an autotrophic organism or by a fermenting organism fermenting fermentable sugars from one or more sugar crops, starch-containing and lignocellulose-containing materials.

3. The method according to claim 2, wherein the volatile fermentation product is one or more selected from ethanol, 1-propanol, 2-propanol, n-butanol, sec-butanol, iso-butanol, acetone, acetate, butyrate, acetaldehyde, acetoin, 2,3-butanediol, butanone, and flavouring ester compounds.

4. The method according to claim 1, wherein the volatile fermentation product is produced by fermentation.

5. The method according to claim 1, wherein the heat recovered by the heat recovery apparatus is used for preheating of air or liquids for a commercial process, general heating of hot water for industrial or household use, or for generating power utilizing a Rankine cycle, an organic Rankine cycle, or any modification thereof.

6. The method according to claim 1, wherein extractive fermentation is utilized during the solar-assisted volatile fermentation product production process.

7. An apparatus for recapturing heat from a solar-assisted volatile fermentation product production process comprising a heat recovery apparatus and a solar-assisted volatile fermentation product production apparatus.
